(19) United States
(12) Patent Application Publication
(10) Pub. No.: US 2007/0156110 A1
(43) Pub. Date: Jul. 5, 2007

(54) Diaper with baffle overflow protection

(76) Inventor: Kevin Thyfault, Towanda, KS (US)

Correspondence Address: Bradley P. Sylvester, Suite 300, 200 North Broadway, Wichita, KS, 67202, US

(21) Appl. No.: 11/326,186

(22) Filed: Jan. 5, 2006

(51) Int. Cl. A61F 13/15

(52) U.S. Cl. 604/385.101; 604/385.19

(57) ABSTRACT

An improved diaper or pull up type pant, using attached side walls that are defined from the front to the back of the diaper or pull up type pant, with one or more baffles positioned between said side walls so as to define multiple cavities for the collection and retention of waste. The multiple cavities provide additional barriers, and assist in preventing waste product from moving out of the front top or rear top portion of the pull up type pant or diaper.

Improved diaper with baffle overflow protection

This is not a continuation in part of any other application.

No Federally sponsored research or development.

No incorporation-by-reference materials are included with this application

### BACKGROUND OF THE INVENTION

Infants and toddlers often have bowel movements that exceed the capacity of the diaper or pull up type pant to contain, due to insufficient barriers to prevent leakage. For this reason, disposable diapers or pull up type pants often are created with features that are intended to increase the absorption or holding capacity with regard to excessive amounts of waste products. Features such as the elastic leg openings, and top flaps folded inward assist but do not wholly prevent leakage. Cloth diapers, by comparison, suffer from the inability to include any such additional features, and are limited to their absorption capability only. It is not an uncommon sight for a small child to have a large bowel movement, to the extent that the runny fecal matter or diarrhea either escapes through the leg openings, or is more often forced out at the top back opening between the diaper or pull up type pant and the child's lower back. When this happens, the clothing becomes soiled, and the inconvenience and overall mess increases substantially.

Efforts have been made to try and prevent runny fecal matter or diarrhea from exiting out of the top back side or front top side of a diaper or pull up type pant. Diapers and pull-ups have been provided with elastic waistbands, which serve to provide a restrictive barrier through the negating of any opening through which fecal matter can flow. Any person familiar with the average child knows and understands well that the child is seldom still, and its movement will eventually cause a gap to be created between the diaper and their lower back area. When this occurs, a failure to contain a voluminous liquid bowel movement will occur. Various improvements in diapers and pull up type pants have been created to prevent this type of diaper failure. However, none have been wholly successful.

U.S. Pat. No. 6,648,870 (Itoh et al.) discloses a disposable diaper where the walls of the diaper are shown to provide leakage protection. These walls function in some respects as a baffle, but do not teach the idea of having the baffles or walls defined across the width of the diaper.

Referring now to U.S. Pat. No. 6,454,747 (Shimada), a disposable diaper is shown that has a flap that folds over, and where the flap helps prevent leakage. A similar type of leakage protection is shown in U.S. Pat. No. 6,083,212 (Kumaska), in which the concept of the “baffle” is indicated. However, both of these patents do not anticipate multiple flaps or baffles across the width of the diaper on the inner side. The prior art teaches that a baffle or flap assists in preventing leakage. The concept of additional baffles or flaps is not taught by the prior art.

U.S. Pat. No. 4,753,646 (Enloe) discloses a diaper that has a flap and at least one fluid waste containment pocket. However, this patent also fails to teach or disclose the present invention of multiple flaps or baffles placed along the width of a diaper. Referring now also to U.S. Pat. No. 3,930,501 (Schaar), an early diaper having an end flap is shown. Such end flaps are quite common, and although some significant attention is given to the flaps, and even mentioned specifically in the claims, there is no indication additional flaps are contemplated.

### BRIEF SUMMARY OF THE INVENTION

This invention comprises an improved disposable diaper, that utilizes existing structure and technology, with the additional adaptations of having one or more baffles placed between typical side walls or flaps that are present on many disposable diapers or pull up type pants. The typical diaper or pull up type pant has an absorbent layer with an exterior that is fairly impervious to the transfer of liquid through it. On the interior side, side walls are usually provided, which provide a barrier to trap waste matter and prevent it from laterally exiting the diaper. Certain types of pull-up pants may also utilize these side walls. The side walls are generally parallel to one another, and are placed on the diaper or pull-up pants so that they travel from the back of the wearer to the front of the wearer, defining a trough to collect excess waste products. Again, many types of pull-up pants and diapers have similar structural similarities.

The present invention further adds one or more baffles, which comprise barriers that extend across the gap or trough area created by the side walls. The baffles should be angled, in relation to the diaper and side wall, or may be perpendicular to the diaper base portion and side wall.

Several baffles may be used with a single diaper or pull-up pants. It may be advantageous to provide multiple baffles that are spaced apart from each other, with the resulting space between the baffles comprising individual collection cavities. Several different collection cavities will assist in providing maximum collection capability, and will better prevents leakage due to a large volume of waste products.

Accordingly, it is the object of this invention to provide an improved leakage protection system for both diapers and the pull up type pants, using multiple cavities.

### DESCRIPTION OF THE DRAWINGS

FIG. 1 is a perspective view of the improved diaper, with the diaper opened so as to reveal the interior portion of it, so that the baffles and various collection cavities are shown.

FIG. 2 is a cross sectional view of the sidewall of a diaper, depicting two baffles attached to it.

FIG. 3 is a side view of a baby wearing the improved diaper or pull-up type pants, showing the various baffles, and their location and orientation in relation to the diaper or pull up type pant.

FIG. 4 is a perspective view of pull-up type pants, showing sidewalls and baffles that are part of the structure of the pants.

### DETAILED DESCRIPTION OF THE INVENTION

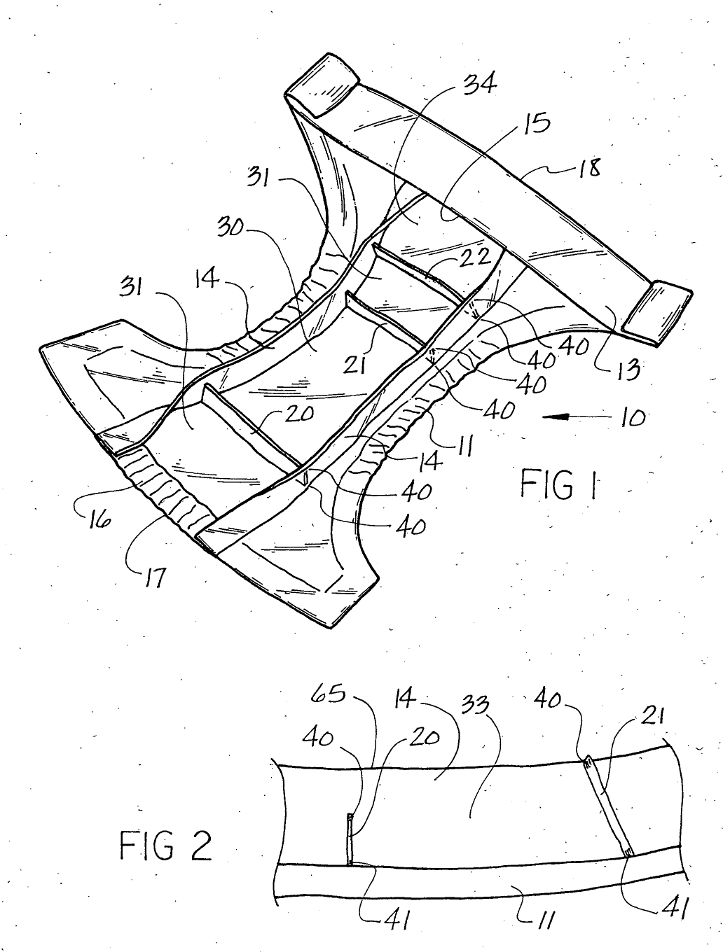

Referring now to FIG. 1, an improved diaper 10 is shown, with the diaper 10 opened so as to reveal the interior side of said diaper 10. The base portion of the diaper 10 comprises an absorbent layer 1, which has an external surface that prevents leakage directly from the interior portion of the diaper 10 through the outer surface of the diaper 10.

The interior surface of the diaper 10 defines a front top edge 17, with elastic band area 16, and front securing flaps 12. This portion of the diaper 10 is worn so that it is adjacent to the front or lower stomach area of the person wearing it. The elastic band 16 is not required, but is typically present on most disposable diapers 10.

The back top edge 18 is adjacent to the wearer's lower back area when worn, and the back securing flaps 13 are attached to the front securing flaps 12 on each of the respective sides, so that the diaper 10 is secured around the waist area of the person wearing it. When worn, a diaper 10 will function essentially as a pull up type pant 50, as shown in FIG. 4.

As FIG. 1 shows, a folded flap 15 may be provided along the back top edge 18, and is commonly done so in previous disposable diapers 10. The folded flap 15 in the past has provided a last line of defense against overflow of liquid waste product, commonly known as diarrhea. A typical diaper, unlike the diaper 10 referred to here, easily succumbs to a failure to contain a large amount of diarrhea. The typical diaper is able to often prevent leakage around the leg openings, due to sidewalls 14, as is shown in FIG. 1, but fails to prevent leakage out the back, since the sidewalls 14 effectively define a continuous collection cavity from the anal area to the top back edge 18, and from the anal area to the front top edge 17.

FIG. 1 depicts sidewalls 14, which comprise generally elastic and flexible walls that are attached to the base absorbent layer 11, and which have an elasticized top 65 which in a relaxed state contracts to a shorter length than the portion attached to the absorbent layer 11. The result is that the elasticized top 65 will generally conform to the wearer's skin surface, and provide a fairly well sealed barrier against leakage.

A drawback to the typical diaper, unlike the diaper 10 shown in FIG. 1, is that the sidewalls 14 are generally parallel to one another and travel from the front top edge 17 to the back edge 18, as shown in FIG. 1. In a typical diaper, the resulting space between the two sidewalls comprises a single trough like collection cavity, and therefore if the person wearing the typical diaper sits down, diarrhea within the trough like collection cavity will be squeezed upward toward the back top edge 18 or front top edge 17. In a typical diaper, the only barrier to prevent leakage out the back side of the diaper is a folded flap 15. As is often the case, and the experience of many parents, this single barrier proves to be unable to handle and cope with any significant amount of diarrhea.

The present invention world is provided with one or more baffles, which serve as barriers across the space defined by the sidewalls 14. Referring again to FIG. 1, a baffle 21 is shown, in which the baffle 21 comprises a sheet of material and that is connected at a first end to one of the sidewalls 14, and connected at the second end to the other side wall 14, with the said barrier 21 and positioned between said side walls 14. The bottom edge of the baffle 21 is fixed to the absorbent layer 111 of the diaper 10, so as to create a seal between the baffle 21 and absorbent layer 11. This structural description for baffle 21 should be understood to be duplicative for any other baffle used with diaper 10.

Referring again to FIG. 1, a second baffle 20 is shown, which is also attached to the diaper 10 in the same way as baffle 21. It should also be understood that baffles 21 and 20 may also be fixed to the absorbent layer 11, in addition to the side walls 14. As a result, a main collection cavity 30 is defined by sidewalls 14, and baffles 20 and 21, and the absorbent layer 11. A diaper 10, using baffles 20 and 21, to define a main collection cavity 30 between the sidewalls, offers greater protection against leakage than a prior art diaper without said baffles 20 and 21.

Additional baffles may be used, to provide additional collection cavities. As is further shown in FIG. 1, the placement of baffle 22 defines a secondary rear collection cavity 33, along with baffle 22 providing a secondary barrier against the movement of diarrhea and towards the back top edge 18. The secondary rear collection cavity 33 can function as an overflow for diarrhea moving out of the main collection cavity 30, and yet keep any further movement toward the back top edge 18 to a minimal level.

It should be understood that FIG. 1 does not limit the number of baffles that may be used. Although FIG. 1 indicates a total of three baffles 20, 21 and 22, more baffles may be used as necessary. FIG. 4 depicts additional baffles 23 and 24 for example. In a diaper 10, such as that shown in FIG. 1, the folded flap 15 may act as a baffle, so as to define an additional overflow cavity of 34, defined by baffle 22, sidewalls 14, and the folded flap 15. The overflow cavity 34 would receive and contain any matter moving past baffle 22 out of the secondary rear collection cavity 33, and restrain and prevent further movement of any diarrhea past the back top edge 18.

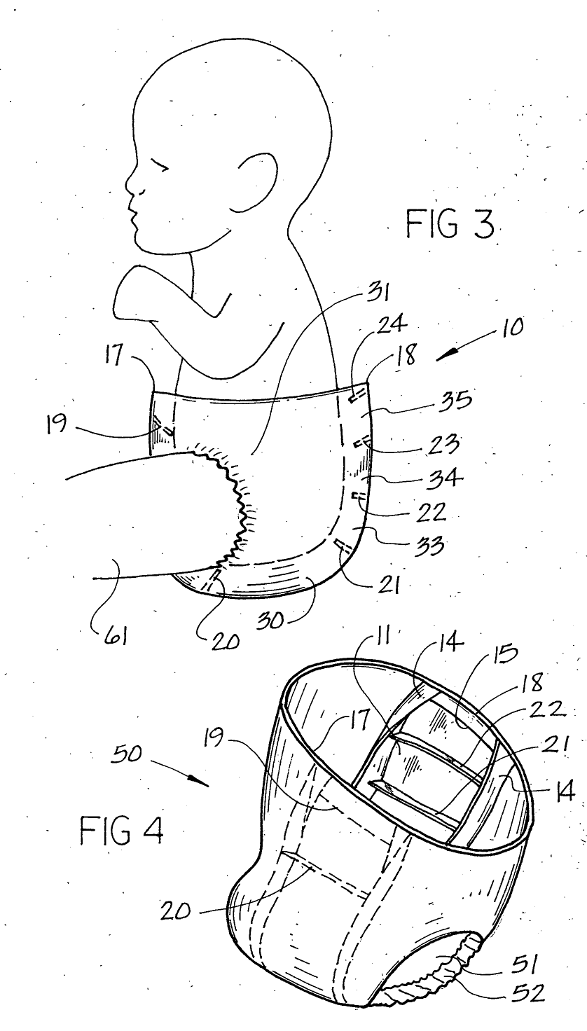

One optimal configuration for the improved diaper 10 is to utilized the side walls 14, with two baffles 20 and 21, so as to define a single main collection chamber 30. This configuration defines a diaper 10 with a collection chamber 30 that is able to hold a quantity of waste effectively. The main collection cavity 30 is defined on the diaper 10 in such a manner that it will be directly below the anal opening of the person wearing the diaper 10. This position for the main collection cavity 30 is exemplified in FIG. 3. It should be also understood that the discussion and description of the diaper 10 with baffles 20 and 21 will also apply to pull up type pant 50, as noted in FIG. 4, and exemplified as to structure in FIG. 3 when worn.

The elastic band 16, on the front portion of the diaper 10, may also serve as a last barrier, and define a secondary front collection cavity of 31, which would contain overflow from the main collection cavity 30 that was moving towards the top front edge 17.

Referring now to FIG. 2, a cross-sectional view of the side walls 14, diaper absorbent layer 11, and baffles 20 and 21 and are shown. Any baffles may be attached to the sidewalls 14 through any means commonly known and understood in the art. One available means, is to cause a stitch of attachment thread to fix the position of the baffle in relation to the side flap 14. As is shown in FIG. 1 and FIG. 2, the various baffles are angled in relation to the side wall 14. The angle of the baffle 20 and 21, for example, will assist in providing a sturdy barrier when presented with diarrhea, through a cupping action that will serve to catch any waste matter. Baffles 20, 21 and 22, or even baffles 19, 23 and 24 as shown in FIG. 4, may be angled differently in relation to each other, depending on where they are positioned along the length of the side walls 14. Each baffle 19-24 is anchored, and may be done so by a stitch 41, and angularly positioned by a top stitch 40. Any attachment means may be used as desired. Moving from the center of the diaper 10, being the main collection cavity 30, any baffle provided should be angled so that the top portion of it is leaning in toward the central main collection cavity 30.

Referring now to FIG. 3, a person 60 is shown wearing the diaper 10, with the person 60 shown in a sitting position, so that their leg 61 extends outward at a right angle to their torso. This invention is directed toward children, but adults and any person needing to wear a diaper or pull up type pant is applicable and suitable for this invention. As is also shown in FIG. 3, a series of baffles is provided, which define various collection cavities. FIG. 3 depicts more baffles than FIG. 1, and as a result more collection cavities are defined.

Continuing to refer to FIG. 3, the diaper 10 is provided with baffles 20 and 21 which define the main collection cavity 30. This cavity 30 would typically be first responsible for maintaining and holding any waste product not absorbed by the diaper's absorbent layer 11. As is shown, baffle 21 is angled toward the main collection cavity 30, and likewise baffle 20 is also angled toward the main collection cavity 30. In the event that waste product manages to move past either baffle 20 or 21, they will move into another collection cavity. If the waste product moves towards the front, past baffle 20, it will then move into a secondary front collection cavity 31, and is therefore further restrained from movement passed the top edge 17 by another baffle 19 and one that is also angled toward the main collection cavity 30.

If waste product moves past baffle 21, it will move into a secondary rear collection cavity 33, and will be restrained against further rear word movement by baffle 22. Baffle 22 is likewise angled toward the main collection cavity 30, and is also angled against the projected flow of waste product moving out a said main collection cavity 30 into secondary rear collection cavity 33.

In the event waste product moves past baffle 22, it will move into an overflow cavity 34, and will be restrained against further movement toward the back top edge 18 by baffle 23. FIG. 3 also indicates an additional baffle 24, that defines an additional collection cavity 35, where the additional collection cavity 35 is defined by the absorbent layer 11, sidewalls 14, baffle 23 and baffle 24.

Referring now also to FIG. 4, a pull up type pant 50 is shown. This type of pant 50 is generally used when a child has moved out of diapers and is in between diapers and regular underwear, with regard to their potty training. The pull up type pant 50 functions very similarly to the diaper 10, when the diaper 10 is being worn. The main difference between a diaper 10 and a pull up type pant 50 is that the pull up type pant 50 must be removed without detaching any flaps. The pull up type pant 50 must be pulled down, or torn at the sides to allow removal. During wear, the baffles and multiple chambers will work identically between the diaper 10 and the pull up type pant 50.

As is shown in FIG. 4, sidewalls 14 are defined from the top back edge 18 to the top front edge 17, with the said side walls 14 defining a fairly linear cavity between them. Various baffles 19, 20 21 and 22 are depicted and shown, and operate the same way as is shown and described in FIG. 3. Elastic edges 52 define leg openings 51. In both the pull up pant 50, and the diaper 10, any baffles used define one or more cavities that provide multiple barriers against the flow of waste matter, where said cavities are defined by the side walls 14, the absorbent layer 11, and any baffles used, in the same manner as the diaper 10 discussion given above.

From the foregoing statements, summary and description in accordance with the present invention, it is understood that the same are not limited thereto, but are susceptible to various changes and modifications as known to those skilled in the art and we therefore do not wish to be limited to the details shown and described herein, but intend to cover all such changes and modifications which would be encompassed by the scope of the appended claims.

I claim:

1. An improved disposable diaper having at least one collection cavity for the collection and storage of waste matter, where said collection cavity prevents waste matter from moving out of the diaper past the front top edge or back top edge, comprising a diaper having:
a. an absorbent layer;
b. two parallel side walls, attached to the absorbent layer, with each side wall attached from the front to the back portion of the diaper, where the side walls define a trough collection cavity;
c. one or more baffles, positioned between said side walls, defining at least one divider within said trough like cavity for collection of waste.

2. An improved disposable diaper having at least one collection cavity for the collection and storage of waste matter, to prevent waste matter from moving out of the diaper past the front top edge or back top edge, as defined in claim 1, in which the baffles are attached to the side walls and also to the absorbent layer.

3. An improved disposable diaper having at least one collection cavity for the collection and storage of waste matter, to prevent waste matter from moving out of the diaper past the front top edge or back top edge, as defined in claim 1, in which the baffles are angled toward a main collection cavity.

4. An improved disposable diaper having at least one collection cavity for the collection and storage of waste matter, to prevent waste matter from moving out of the diaper past the front top edge or back top edge, as defined in claim 1, in which baffles define a main collection cavity, and where multiple baffles are used to define additional collection cavities.

5. An improved disposable diaper having at least one collection cavity for the collection and storage of waste matter, to prevent waste matter from moving out of the diaper past the front top edge or back top edge, as defined in claim 1, in which two baffles are positioned between said sidewalls so as to define a main collection cavity, defined by said sidewall, two baffles, and the absorbent layer.

6. An improved pull up type pant having at least one collection cavity for the collection and storage of waste matter, to prevent waste matter from moving out of the pull up type pant past the front top edge or back top edge, in which said pull up type pant has two side walls, with each side wall attached to said pull up type pant from the front to the back portion of the pull up type pant, with the side walls defining a trough collection cavity, and where at least one baffle is positioned between said sidewalls of said trough like cavity.

7. An improved disposable pull up type pant having at least one collection cavity for the collection and storage of waste matter, to prevent waste matter from moving out of the pull up type pant past the front top edge or back top edge, as defined in claim 6, in which the baffles are attached to the side walls and also to the pull up type pant absorbent layer.

8. An improved disposable pull up type pant having at least one collection cavity for the collection and storage of waste matter, to prevent waste matter from moving out of the pull up type pant past the front top edge or back top edge, as defined in claim 6, in which the baffles are angled toward a main collection cavity.

9. An improved disposable pull up type pant having at least one collection cavity for the collection and storage of waste matter, to prevent waste matter from moving out of the pull up type pant past the front top edge or back top edge, as defined in claim 6, in which baffles define a main collection cavity, and where multiple baffles are used to define additional collection cavities.

10. An improved disposable pull up type pant having at least one collection cavity for the collection and storage of waste matter, to prevent waste matter from moving out of the pull up type pant past the front top edge or back top edge, as defined in claim 6, in which two baffles are positioned between said sidewalls so as to define a main collection cavity, defined by said sidewall, two baffles, and the absorbent layer.
